(19) United States
(12) Patent Application Publication
(10) Pub. No.: US 2024/0366107 A1
(43) Pub. Date: Nov. 7, 2024

(54) APPARATUS AND METHOD FOR CORRECTING TIP TOE WALKING

(71) Applicant: SANJAY VINOTHKUMAR, South Windsor, CT (US)

(72) Inventor: SANJAY VINOTHKUMAR, South Windsor, CT (US)

(21) Appl. No.: 18/143,064

(22) Filed: May 3, 2023

(51) Int. Cl. A61B 5/103; A43B 7/00

(52) U.S. Cl. CPC A61B 5/1036; A43B 7/00

(57) ABSTRACT

Apparatus for initiating a human response to perceived toe walking which includes an elongated a shoe insole dimensioned and configured for placement within the shoe of a human person; the insole having a toe portion and a heel portion at at opposite axial extremities of the shoe insole; a momentary electric switch is disposed proximate to the toe portion, the momentary electric switch is responsive to tip toe stepping by a human wearing a shoe having the elongated insole installed therein. The apparatus also includes a vibrator assembly disposed distal to the toe portion; a battery assembly disposed distal to the toe portion.

### TECHNICAL FIELD

The present invention is generally directed to therapeutic methods and apparatus. More particularly, the present invention is directed to a method and apparatus for correcting mild tip toe walking. Toe walking is by definition when a person walks on his or her toes or does not contact the ground with his or her heel during a step. Even more particularly, the present invention is directed, in some forms to method and apparatus for treatment of the symptoms of mild cerebral palsy.

### BACKGROUND OF THE INVENTION

Spasticity is a condition in which muscles stiffen or tighten, preventing normal fluid movement. The muscles remain contracted and resist being stretched, thus affecting movement, speech and gait. Spasticity is generally caused by damage or disruption to the area of the brain and spinal cord that are responsible for controlling muscle and stretch reflexes. These disruptions can be due to an imbalance in the inhibitory and excitatory signals sent to the muscles, causing them to lock in place. Spasticity can be harmful to growing children as it can affect muscles and joints. People with brain injury, spinal cord injury, cerebral palsy or multiple sclerosis can have varying degrees of spasticity.

Symptoms of spasticity can vary from being mild stiffness or tightening of muscles to painful and uncontrollable spasms. Pain or tightness in joints is also common in spasticity as well as muscle stiffness, causing movements to be less precise and making certain tasks difficult to perform. Sometimes muscle spasms, cause uncontrollable and often painful muscle contractions

The human brain has a capacity to continue growing and evolving in response to life experiences. Plasticity is the capacity to be shaped or altered; neural plasticity, then, is the ability for the brain to adapt or change over time, by creating new neurons and building new networks.

The importance of neuroplasticity cannot be overstated. It means that it is possible to change dysfunctional patterns of thinking and behaving and to develop new mindsets, new memories, new skills and new abilities.

Stimulating neuroplasticity by enhancing synaptic connectivity through any variety of means actively promotes cognitive and mental health and blunts the impact of negative stimuli. One approach to stimulating neuroplasticity is to provide a method and apparatus to immediately notify the user when a given event occurs so that the user can adapt his or her behavior to medically preferable.

From the above, it is therefore seen that there exists a need in the art to overcome the deficiencies and limitations described herein and above.

### SUMMARY OF THE INVENTION

The shortcomings of the prior art are overcome and additional advantages are provided through apparatus for initiating a human response to perceived toe walking which includes an elongated a shoe insole dimensioned and configured for placement within the shoe of a human person; the insole having a toe portion and a heel portion at at opposite axial extremities of the shoe insole; a momentary electric switch is disposed proximate to the toe portion, the momentary electric switch is responsive to tip toe stepping by a human wearing a shoe having the elongated insole installed therein. The apparatus also includes a vibrator assembly disposed distal to the toe portion; a battery assembly disposed distal to the toe portion. The momentary electric switch, the vibrator and the battery assembly are connected in series whereby momentary closure of the momentary electric switch causes the vibrator assembly to vibrate.

The present invention also includes a method for initiating a human response to perceived toe walking which includes providing an elongated a shoe insole dimensioned and configured for placement within the shoe of a human person and having a toe portion and a heel portion at at opposite axial extremities of the shoe insole; providing a momentary electric switch disposed proximate to the toe portion, the momentary electric switch being responsive to tip toe stepping by a human wearing a shoe having the elongated insole installed therein, providing a vibrator assembly disposed distal to the toe portion; a battery assembly disposed distal to the toe portion; and connecting the momentary electric switch, the vibrator and the battery assembly in series whereby momentary closure of the momentary electric switch causes the vibrator assembly to vibrate. The method may further including providing a shoe dimensioned and configured for receiving the insole.

Additional features and advantages are realized through the techniques of the present invention. Other embodiments and aspects of the invention are described in detail herein and are considered a part of the claimed invention.

The recitation herein of desirable objects which are met by various embodiments of the present invention is not meant to imply or suggest that any or all of these objects are present as essential features, either individually or collectively, in the most general embodiment of the present invention or in any of its more specific embodiments.

### BRIEF DESCRIPTION OF THE DRAWINGS

The subject matter which is regarded as the invention is particularly pointed out and distinctly claimed in the concluding portion of the specification. Although specific features of various exemplary embodiments of the invention may be shown in some drawings and not in others, this is for convenience only. In accordance with the principles of the invention, any feature of a drawing maybe referenced and/or claimed in combination with any feature of any other drawing.

The invention, however, both as to organization and method of practice, together with the further objects and advantages thereof, may best be understood by reference to the following description taken in connection with the accompanying drawings in which:

FIG. 1 is a partly schematic view of a human foot A disposed within a shoe together within an insole in accordance with one form of the present invention,

FIG. 2 is a partly schematic view showing the bottom view of the right foot insole in greater detail.

FIG. 3 is a partly schematic view showing a bottom view of the left foot insole in greater detail.

### DETAILED DESCRIPTION

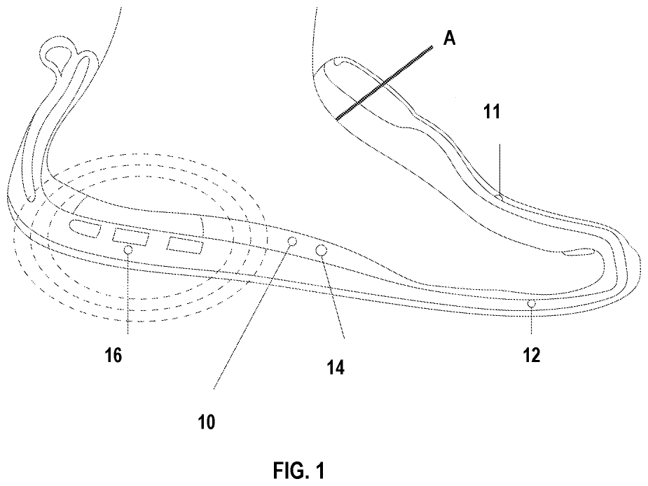

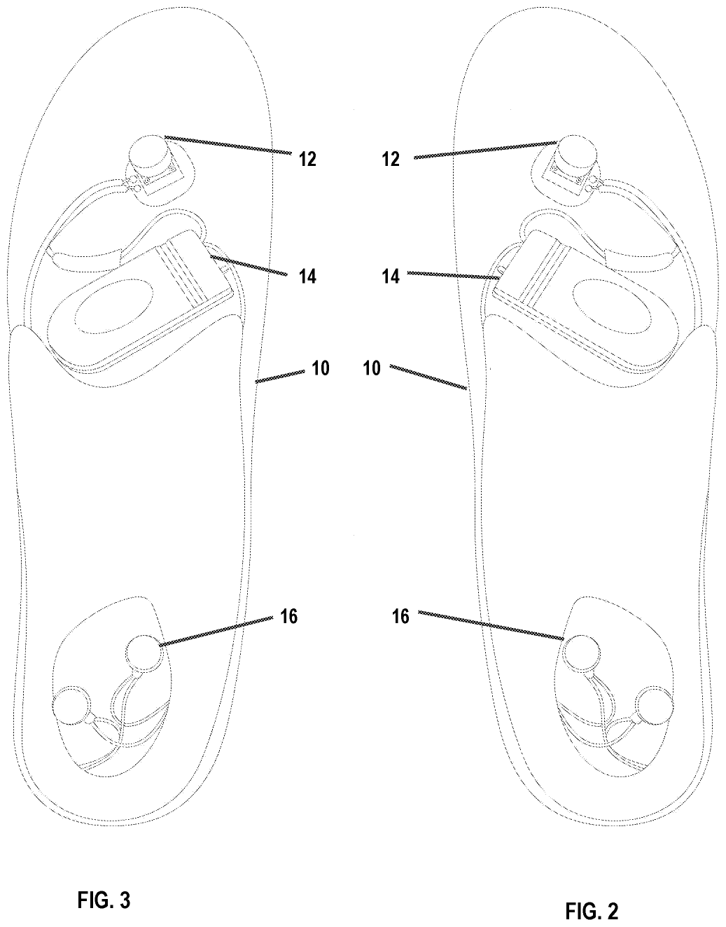

Referring now to FIG. 1 of the drawing there is shown a human foot A within a shoe 11 having an insole 10. FIGS. 2 and 3 illustrate left and right insoles in accordance one form of the present invention. The particular patient conduct to be addressed is walking on tippy toes. Accordingly, each insole 10 is dimensioned and configured for engagement with respective left and right shoes 11 (one shown.)

Disposed proximate to the toe end of each insole is a momentary switch 12. A momentary switch by definition, conducts electric power when a compressive force is concurrently placed on the top and bottom of the switch 12.

The momentary switch 12 is part of a series circuit that also includes a battery case with an internal battery 14 and a vibrator 16. Closure of the switch 12 allows electrical power from battery case with an internal battery 14 to operate the vibrator 16.

Accordingly, when a user walks in a tip toe manner a compressive force between the user's foot and the ground causes momentary closure of a switch 12 to deliver electric power from the battery case with an internal battery 14 to the vibrator 16. Thus, the user is given an immediate stimulus whenever the same conduct occurs. The repetitive stimulus in response to assists the user to take corrective action.

More particularly, the consequent vibration serves to remind the user that a tip toe step has been started and to adjust his or her manner of stepping.

Learning programs the brain. It is nature's way to create simultaneously both “hardware” and “software” for the brain. Neuroscientists have long known that learning experiences change the functionality cover circuitry that is used to process and remember a given learning event. The circuit changes anatomical. Electron microscope photographs reveal that the synaptic changes take the form of little blebs located on dendrites. These blebs our called “dendritic spines,” in their size and number change in response to learning and memory formation.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

It will be understood that, in general, terms used herein, and especially in the appended claims, are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended such an intent will be explicitly recited in the claim, and in the absence of such recitation no such intent is present. For example, as an aid to understanding, the following appended claims may contain usage of introductory phrases such as “at least one” or “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “an imager” should typically be interpreted to mean “at least one imager”); the same holds true for the use of definite articles used to introduce claim recitations. In addition, even if a specific number of an introduced claim recitation is explicitly recited, it will be recognized that such recitation should typically be interpreted to mean at least the recited number (e.g., the bare recitation of “two images,” or “a plurality of images,” without other modifiers, typically means at least two images). Furthermore, in those instances where a phrase such as “at least one of A, B, and C,” “at least one of A, B, or C,” or “an [item] selected from the group consisting of A, B, and C,” is used, in general such a construction is intended to be disjunctive (e.g., any of these phrases would include but not be limited to systems that have A alone, B alone, C alone, A and B together, A and C together, B and C together, or A, B, and C together, and may further include more than one of A, B, or C, such as A₁, A₂, and C together, A, B₁, B₂, C₁, and C₂ together, or B₁ and B₂ together). It will be further understood that virtually any disjunctive word or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Although the description above contains many specifics, these should not be construed as limiting the scope of the invention, but as merely providing illustrations of some of the presently preferred embodiments of this invention. Thus, the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

While the invention has been described in terms of various specific embodiments, those skilled in the art will recognize that the invention can be practiced with modification within the spirit and scope of the claims. While certain embodiments of the invention have been illustrated and described, various modifications are contemplated and can be made without departing from the spirit and scope of the invention. Accordingly, it is intended that the invention is not to be limited, except as by the appended claim(s).

The teachings disclosed herein may be applied to other systems and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the present invention with which that terminology is associated. In general, the terms used in the following claims should not be constructed to limit the present invention to the specific embodiments disclosed in the specification unless the above description section explicitly define such terms. Accordingly, the actual scope encompasses not only the disclosed embodiments, but also all equivalent ways of practicing or implementing the disclosed present invention. The above description of embodiments of the present invention is not intended to be exhaustive or limited to the precise form disclosed above or to a particular field of usage.

While certain aspects of the present invention are presented below in particular claim forms, various aspects of the present invention are contemplated in any number of claim forms. Thus, the inventor reserves the right to add additional claims after filing the application to pursue such additional claim forms for other aspects of the present invention.

What is claimed is:

1. Apparatus for initiating a human response to perceived toe walking which comprises:
an elongated a shoe insole dimensioned and configured for placement within the shoe of a human person;
said insole having a toe portion and a heel portion at opposite axial extremities of the shoe insole;
a momentary electric switch disposed proximate to said toe portion, said momentary electric switch being responsive to tip toe stepping by a human wearing a shoe having said elongated insole installed therein,
a vibrator assembly disposed distal to said toe portion; and
a battery assembly disposed distal to said toe portion;
said momentary electric switch, said vibrator and said battery assembly being connected in series whereby momentary closure of said momentary electric switch causes said vibrator assembly to vibrate.

2. The apparatus as described in claim 1 further including a shoe dimensioned and configured for receiving said insole.

3. A method for initiating a human response to perceived toe walking which comprises:
providing an elongated a shoe insole dimensioned and configured for placement within the shoe of a human person;
providing an insole having a toe portion and a heel portion at at opposite axial extremities of the shoe insole;
providing a momentary electric switch disposed proximate to said toe portion, said momentary electric switch being responsive to tip toe stepping by a human wearing a shoe having said elongated insole installed therein,
providing a vibrator assembly disposed distal to said toe portion;
a battery assembly disposed distal to said toe portion; and
connecting said momentary electric switch, said vibrator and said battery assembly being connected in series whereby momentary closure of said momentary electric switch causes said vibrator assembly to vibrate.

3. The method as described in claim 3 further including providing a shoe dimensioned and configured for receiving said insole.
